(19) United States
(12) Patent Application Publication
(10) Pub. No.: US 2012/0118300 A1
(43) Pub. Date: May 17, 2012

(54) FIXATION BANDAGE FOR THE FIXATION OF A PATIENT

(76) Inventor: Roman Wysozki, Hamburg (DE)

(21) Appl. No.: 13/297,310

(22) Filed: Nov. 16, 2011

(30) Foreign Application Priority Data
Nov. 16, 2010 (EP) 10 014 658.8

(51) Int. Cl. A61F 5/37

(52) U.S. Cl. 128/876

(57) ABSTRACT

A fixation bandage for fixing a patient on a bed or another repository, having a body part belt to be closed around a body part of the patient in a ring-shaped fashion. The bandage further has a bed belt, flatly to be put onto a reclining surface of the bed and having a bed holder on each of its two ends for fastening it on a bed frame of the bed, and on which the body part belt is fastened. The fixation bandage is further characterised in that, for additional lateral fastening of the body part belt, the bed holders are each one formed by one fastening belt, whose length is dimensioned such that starting from one of the ends of the bed belt, it can be guided around a beam of the bed frame and can be guided back to the body part belt and is realised to be fastened on the body part belt.

### BACKGROUND OF THE INVENTION

The present invention relates to a fixation bandage for the fixation of a patient on a bed or another repository.

A fixation bandage known for this purpose has a bed belt to be laid flatly onto the bed and having a bed holder on each of its two ends, for fastening it on a bed frame of the bed. The bed holders are each wound around a beam of the bed frame in the form of a loop, and are closed with a suitable locking device, a magnetic lock for instance. Through this, the bed belt is fastened on the bed. A waist belt is fastened on the bed belt, which is circularly laid around the waist of the patient and can be closed by a further locking device. Through this, the patient is fixed on the bed at the waist. This protects the patient from falling out of the bed, without unnecessarily limiting his/her freedom of movement, of the hands and feet in particular. Such a fixation bandage is described in the utility DE 203 17 701 U1.

In order to achieve better fixation of the patient, a fixation bandage with an additional fixation belt which is fastened on the waist belt and at the side on the bed holders is known from the utility DE 20 2006 009 390 U1.

The fixation bandage described in the utility DE 203 17 701 U1 has two lateral fastening devices fixedly connected to the waist belt for additional lateral fastening, whose free ends are locked on the bed holders. These lateral fastening devices can reliably prevent a patient from turning himself to the side. This is absolutely necessary in many cases for safety reasons, in order to prevent the chest from any lateral leaning out or slipping out from the bed, which can result in injuries through the waist belt.

Starting from this, it is the objective of the present invention to provide a fixation bandage which permits a safe fixation of a patient in the practical utilisation.

### SUMMARY OF THE INVENTION

This objective is achieved by a fixation bandage for the fixation of a patient on a bed or another repository with the features of claim 1. Advantageous embodiments are indicated in the subsequent subclaims.

The fixation bandage has
a body part belt to be closed around a body part of the patient in a ring-shaped fashion,
a bed belt, flatly to be put onto a reclining surface of the bed and having a bed holder on each of its two ends for fastening it on a bed frame of the bed, and on which the body part belt is fastened, wherein
for additional lateral fastening of the body part belt, the bed holders are each one formed by one fastening belt, whose length is dimensioned such that starting from one of the ends of the bed belt, it can be guided around a beam of the bed frame and can be guided back to the body part belt, and is realised to be fastened on the body part belt.

The present invention is based on the finding that in fact, the known fixation bandages mentioned in the beginning permit a safe fixation in principle, but are not always correctly applied in practice. The reason for this is that a certain fixation of the patient is already achieved by the fixation of the bed belt and the closure of the waist belt around the patient. However, for complete and sufficiently safe fixation, additional fastening of the lateral fastening devices is imperatively necessary. However, this may be left undone in utilization day-to-day practice, due to the work overload of caregivers.

Here, the present invention provides remedy by realising the bed holders as fastening belts which additionally serve for the fixation of the bed belt on the bed for laterally fastening the body part belt. For this purpose, each of the two fastening belts is guided around a beam of the bed frame and fastened on the body part belt. Since fixation of the fixation bandage on the bed takes place only through this fastening, there is no longer a danger that a fastening of conventional, separate lateral fastening devices is omitted. By fastening the fastening belts on the body part belt, there is a fastening of the bed belt on the bed frame and the sidewise fastening at the same time. In particular, in the present invention, no separate fastening of the fixation bandage on the bed frame is necessary in addition to guiding the fastening belts around the beams of the bed frame and fastening the fastening belts on the body part belt. Hence, fixation of the entire arrangement on the bed takes place simply by fastening the fastening belts on the body part belt.

A further advantage of the present invention is that an already existing fixation bandage which still does not have any or only conventional, separate lateral fastening devices, can be upgraded in a simple way according to the present invention. For this purpose, the existing fixation bandage can be equipped with bed holders formed according to the present invention.

The fixation bandage of the present invention serves for the fixation of a patient on a bed or another repository. On the one hand, by fixation is meant to keep patients quiet who are bewildered, uncooperative or not certifiably sane. Moreover, support of patients in a preferred posture for other reasons, for instance decubitus prevention, is also included. The other repository may be for instance a stretcher or a couch, for instance for a patient's transportation. As far as in the features of the claims, reference is made to elements of the bed, like the bed frame or a beam of the bed frame, for the sake of simplicity this is done pars pro toto for corresponding components of other repositories. For instance, in the case of a stretcher, the bed frame may be a frame of the stretcher, and the beam of the bed frame a lateral carrier of this frame.

The bed belt is flatly laid onto a reclining surface of the bed, for instance onto a mattress. Bed holders follow both ends of the bed belt and are formed by sections of the fastening belts. A further section of each fastening belt on each side forms a lateral fastening device.

In utilization practice, the body part belt encloses a body part of the patient, the waist for instance. It may be realised to be relatively broad and upholstered, as the case may be, in order to provide for a high comfort after being applied. It may have a possibility of adjustment, for instance in the form of ears that are spaced apart in the circumferential direction, through which a locking device can be guided through. Through this, the body part belt can always be applied sufficiently tightly without being too uncomfortable.

The body part belt is fastened on the bed belt. For instance, it can be immediately sewn to the bed belt, in particular along a central line of the bed belt and the body part belt running transversely to the longitudinal direction of the belts. The connection may also extend over a longitudinal section of the body part belt and of the bed belt. The fastening of the body part belt on the bed belt may also take place with the aid of furthers belts, which extend over a longitudinal section of the body part belt and the bed belt and are essentially connected with one of the two belts on each of their ends. Such belts can be arranged crosswise, so that the body part belt that is closed in a ring-shaped fashion can be unwound for a certain length on the bed belt, in order to permit a limited rotational movement of the patient.

In practice, each of the two fastening belts extends from one end of the bed belt in the lateral direction at first, towards one beam of the bed frame (or, for instance, towards a lateral carrier of a frame of a stretcher), around the beam and back to the body part belt. It is realised for fastening it on the body part belt, i.e. for instance it has further ears that are suitable for this and are fastened on the body part belt with a locking device. The fastening of the fastening belts on the body part belt takes place in particular on sections of the body part belt which are arranged significantly above the reclining surface in the utilisation, for instance between those positions of the closed body part belt on the upper side thereof which are disposed farthermost laterally at the outside in the utilisation.

In one embodiment, the bed belt and the two fastening belts are formed by one single belt. In principle, the bed belt may be another belt that is connected to the fastening belt. The bed belt, which is situated below the patient in the utilisation, can also have a greater width than the fastening belts, in order to prevent twisting of the bed belt. The fastening belts can be sewn to the bed belt. According to the embodiment, bed belt and fastening belt are formed by one single belt, so that there is no connection of sections that form the fastening belts and the bed belt. This permits a particularly simple production of the fixation bandage. Optionally, a widening or a pad may be provided in the section of the single belt forming the bed belt. However, this is not necessary, because the belt normally lies on the upholstered reclining surface anyway.

In one embodiment, only one single locking device is provided for closing the body part belt and for fastening the fastening belts on the body part belt. In principle, separate locking devices can be used for closing the body part belt and for fastening the two bed belts on the body part belt in the invention, so that for instance three locking devices are necessary for correctly applying the fixation bandage. However, both fastening belts can be fastened on the body part belt by a common locking device, wherein a separate locking device is still used for closing the body part belt. Preferably, only one single locking device is used, however, which then closes the body part belt and fastens the two fastening belts on the body part belt at the same time. Thus, a particularly cost-saving and materials-saving fixation bandage is provided. Only one single locking device is necessary, a magnetic lock for instance. Even the belts can be realised more simply, because separate ears for accommodating the further locking devices are not needed, for instance. Moreover, application of the fixation bandage and in particular the opening thereof is simplified because only one single locking device has to be closed or opened, respectively.

In one embodiment, the locking device is a magnetic lock. The utilisation of magnetic locks for locking fixation bandages is known. It is a locking solution that is particularly simple to handle but safe anyhow. The magnetic lock can normally be closed without a special tool, just by simply plugging together. It can also be opened in a simple manner with the aid of a magnetic key.

In one embodiment, the magnetic lock has a locking button and a base with a disc and a shaft, wherein the length of the shaft is dimensioned such that it can be guided through two ears of the body part belt and through one ear of each fastening belt, and the locking button can be put onto the shaft above the belts. The disc of the base is, for instance, a circular or rectangular base plate, which is normally disposed below the lowermost belt. The risk of injury is counter-acted by a relative large disc. For instance, the disc can be held by a pocket on the lowermost belt, that is sewn on the downside of the lowermost belt. The shaft is then guided sideways, into the pocket and through an ear of the lowermost belt, until the disc sits close on the belt. The base is secured by the pocket to prevent it from slipping out of the ear. The lowermost belt is preferably an end of the body part belt. The belt layer laying above it in the utilisation may be the other end of the body part belt in particular, which has also at least one ear through which the shaft is guided through. The shaft is subsequently guided through one ear of each of the two fastening belts, or openings of the belts are put on the shaft, respectively. Through the length of the shaft which is adapted to the altogether four layers of the different belts, the end of the shaft remote from the disc subsequently projects from the uppermost belt, so that the locking button can be put up. Altogether, correct application of the fixation bandage and reliable and safe fixation of the patient is achieved by one single magnetic lock in a simple way.

In one embodiment, on each of the sections of the fastening belts or of the bed belt that lie on the reclining surface between one of the beams of the bed frame and the body part belt in the utilisation of the fixation bandage, one ring or one loop is arranged, through which the fastening belt can be guided after having been guided around the beam of the bed frame, so that it is held near to the reclining surface. The fastening belt can be freely movable in the ring or in the loop. The ring may be a rectangular metal ring, for instance. The loop can be formed by a piece of belt material that is sewn into a loop, for instance. In both cases, the fastening belt is pulled down to the reclining surface by being guided through, whereby a greater free space below the section of the fastening belt forming the lateral fastening device is avoided. Thus, it prevents the patient from getting a body part caught in the mentioned belt sections, the head or the neck in particular. In this way, danger of injury is counter-acted by simple means.

In one embodiment, on each of the sections of the of the body part belt that are arranged at the side of the body part in the utilisation of the fixation bandage, one ring or one loop is arranged through which one of the fastening belts can be guided through, so that the fastening belts are held near to the body part belt by the rings or loops. The rings or loops can be made as described above. Even in this embodiment, it is achieved to close a free space, in this case that between the sections of the fastening belts forming the lateral fastening devices and the body part belt, here by pulling the fastening belts towards the body part belt. The safety of the fixation bandage is further increased thereby.

In one embodiment, plural spaced apart rings or loops are arranged on the bed belt and on the sections of the fastening belts and/or of the body part belt through which the fastening belt can be guided after having been guided around the beam of the bed frame. For instance, two or more rings or loops can be arranged on each of the sections of the fastening belts and/or on the bed belt which form the bed holders. Thereby the sections of the fastening belts forming the lateral fastening devices are guided even more reliably near to the reclining surface or the body part belt, respectively. This counter-acts the mentioned danger of injury even further.

In one embodiment, at least one of the loops is formed in the form of a hose. In this case, the sections of the fastening belts forming the lateral fastening devices are guided through the hose-shaped loops, so that they are guided in a relatively long longitudinal section. This counter-acts the mentioned danger of injury even further too.

In one embodiment, the body part belt has a Velcro® hook and loop fastener by which the ends of the body part belt can be connected in a ring shaped fashion. This permits a preliminary fixation of the body part belt in a condition enclosed around the body part, and is very helpful in particular when the body part belt is to be closed with the same locking device by which the fastening belts are to be fastened on the body part belt also. Unintended slipping of the body part belt during the application of the fastening belts and before definitive locking is avoided through this.

In one embodiment, Velcro® (hook and loop fastener) elements permitting a preliminary fixation of the fastening belts on the body part belt are arranged on an outer side of the body part belt and on those sides of the fastening belts that face the outer side in the utilisation. The Velcro® fastener elements are a Velcro® fleece and a Velcro® hook in particular. This embodiment makes the application of the fixation bandage easier, because slipping of the fastening belts is prevented until the locking device is closed.

In one embodiment, the body part belt is a waist belt, a chest belt, a thigh belt, a wrist belt, an ankle belt or a head belt for enclosing the respective body part. Additional lateral fastening devices have proven to be useful for waist belts in particular. However, they make sense for the other mentioned belts too, in order to achieve optimum fixation. The present invention is suitable for the mentioned kinds of belts in equal degree.

In one embodiment, the body part belt is a waist belt and the fixation bandage has a crotch strap or a thigh holder and/or a shoulder holder. Through the additional elements, the slipping out of the patient from the waist belt towards the upside or the downside is prevented in particular.

In one embodiment, the body part belt is a waist belt and the fixation bandage has wrist weights or loops for fastening wrist weights. The wrist weights or loops can be arranged and fastened on the bed belt or on the fastening belts in particular. They permit additional fixation of the hands or arms, respectively, of the patient in a particularly simple way.

The present invention will be explained in more detail by way of two examples of its realisation depicted in figures.

### BRIEF DESCRIPTION OF THE DRAWINGS

FIG. 1 shows a fixation bandage of the present invention in the applied state, in a simplified cross sectional depiction.

FIG. 2 shows an alternative of a detail depicted in FIG. 1.

In both figures, the same reference signs are used for corresponding parts.

### DETAILED DESCRIPTION OF THE INVENTION

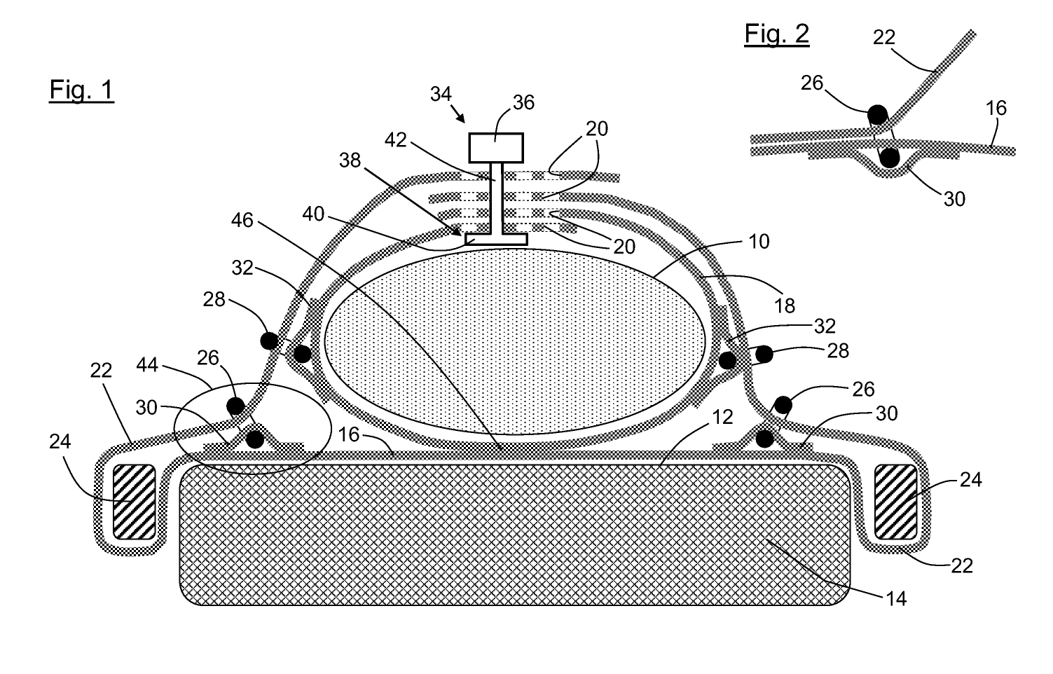

FIG. 1 shows the waist 10 of a patient, which lays on a reclining surface 12 of a mattress 14 of a bed. The patient is fixed by a fixation bandage of the present invention, which has a bed belt 16 laying on the reclining surface 12 and which is made of a textile belt material like the other belts also. The bed belt 16 extends approximately over the entire width of the reclining surface 12, and it has two ends, which are situated near to the edges of the mattress 14.

In the middle of the bed belt 16, a waist belt 18 is fastened at 46, which ring-like encloses the waist 10 of the patient. The waist belt 18 is a body part belt for the body part, “waist,” of the patient. On each one of the two ends of the waist belt 18 are arranged plural ears 20, at least two of the ears 20 laying one above the other in the ring-like closed condition of the waist belt 18.

The fixation bandage is closed by one single magnetic lock 34. The magnetic lock 34 has a locking button 36 and a base 38 with a disc 40 and a shaft 42. The disc 40 is situated below the lower end of the waist belt 18, and the shaft is guided through an ear 20 of each of the four belt layers. Above the uppermost belt layer, which is formed by the fastening belt 22 arranged at the left side in the example, the locking button 36 is plugged up.

Each end of the bed belt 16 is followed by a fastening belt 22, which is guided around a beam 24 of the bed frame, starting from the respective end of the bed belt 16, and guided back to the waist belt 18 from there. Specifically, starting from the beam 24, the fastening belt 22 extends through two rectangular rings 26, 28 made of metal, and from there further in parallel to the waist belt 18 up to an upper side of the waist belt 18, where the two ends of the waist belt 18 lay over the ears 20. Even the free end of each fastening belt 22 has ears 20, from which at least one is arranged above an ear 20 of the waist belt.

Each of the rings 26 is fastened on the upper side of the bed belt 16 with a piece of belt material 30, namely in a section situated sideways between the waist belt 18 and the beam 24 of the bed frame in the utilisation. The two rings 28 are each fastened on one side of the waist belt 18, also with a piece 32 of belt material.

FIG. 2 shows an alternative for the section designated with 44 in FIG. 1. A ring 26 is also shown, which is fastened on the bed belt 16 and through which a fastening belt 22 is guided. In difference to FIG. 1, in the alternative depicted in FIG. 2, the bed belt 16 itself runs also through the ring, and a sewed up piece of belt material 30 is situated on the lower side of the bed belt 16. It serves for fixing the ring 26 on the desired position of the bed belt 16.

1. A fixation bandage for fixing a patient on a bed or another repository, comprising:
a body part belt to be closed around a body part of the patient in a ring-shaped fashion;
a bed belt (16), flatly to be put onto a reclining surface (12) of the bed and having a bed holder on each of its two ends for fastening it on a bed frame of the bed, and on which the body part belt is fastened,
wherein, for additional lateral fastening of the body part belt, the bed holders are each one formed by one fastening belt (22), whose length is dimensioned such that starting from one of the ends of the bed belt (16), it can be guided around a beam (24) of the bed frame and can be guided back to the body part belt and is realised to be fastened on the body part belt.

2. The fixation bandage according to claim 1, wherein the bed belt (16) and the two fastening belts (22) are formed by one single belt.

3. The fixation bandage according to claim 1, wherein one single locking device is provided for closing the body part belt and for fastening the fastening belts (22) on the body part belt.

4. The fixation bandage according to claim 3, wherein the locking device is a magnetic lock (34).

5. The fixation bandage according to claim 4, wherein the magnetic lock (34) has a locking button (36) and a base (38) with a disc (40) and a shaft (42), wherein the length of the shaft (42) is dimensioned such that it can be guided through two ears (20) of the body part belt and through one ear (20) of each fastening belt (22), and the locking button (36) can be put onto the shaft (42) above the belts.

6. The fixation bandage according to claim 1, wherein each of the sections of the fastening belts (22) or of the bed belt (16) that lie on the reclining surface (12) between one of the beams (24) of the bed frame and the body part belt in the utilisation of the fixation bandage, at least one ring (26) or at least one loop is arranged, through which the fastening belt (22) can be guided after having been guided around the beam (24) of the bed frame, so that it is held near to the reclining surface (12).

7. The fixation bandage according to claim 1, wherein, on each of the sections of the body part belt that are arranged at the side of the body part in the utilisation of the fixation bandage, one ring (28) or one loop is arranged through which one of the fastening belts (22) can be guided through, so that the fastening belts (22) are held near to the body part belt by the rings or loops.

8. The fixation bandage according to claim 6, further comprising plural spaced apart rings (26, 28) or loops that are arranged on the bed belt (16) and on the sections of the fastening belts (22) or of the body part belt through which the fastening belt (22) can be guided after having been guided around the beam (24) of the bed frame.

9. The fixation bandage according to claim 6, wherein at least one of the loops is formed in the form of a hose.

10. The fixation bandage according to claim 1, wherein the body part belt has a hook and loop fastener by which the ends of the body part belt can be connected in a ring-shaped fashion.

11. The fixation bandage according to claim 1, wherein the hook and loop fastener elements are arranged on an outer side of the body part belt and on those sides of the fastening belts (22) that face the outer side in the utilisation, allowing a preliminary fixation of the fastening belts (22) on the body part belt.

12. The fixation bandage according to claim 1, wherein the body part belt is a waist belt (18), a chest belt, a thigh belt, a wrist belt, an ankle belt or a head belt for enclosing the respective body part.

13. The fixation bandage according to claim 1, wherein the body part belt is a waist belt (18) and the fixation bandage has a crotch strap, a thigh holder or a shoulder holder.

14. The fixation bandage according to claim 1, wherein the body part belt is a waist belt (18) and the fixation bandage has wrist weights or loops for fastening wrist weights.

15. The fixation bandage according to claim 2, wherein one single locking device is provided for closing the body part belt and for fastening the fastening belts (22) on the body part belt.
